(19) United States
(12) Patent Application Publication
(10) Pub. No.: US 2022/0023182 A1
(43) Pub. Date: Jan. 27, 2022

(54) STABLE ASCORBIC ACID SOLUTION WITH HIGH WATER CONTENT

(71) Applicant: Avon Products, Inc., Suffern, NY (US)

(72) Inventors: Cody N. LEEGWATER, Pompton Lakes, NJ (US); Christopher D. CORINTHIAN, Mahwah, NJ (US); Pradeep H. YADAV, Cliffside Park, NJ (US)

(21) Appl. No.: 17/497,774

(22) Filed: Oct. 8, 2021

Related U.S. Application Data
(63) Continuation of application No. PCT/US20/22224, filed on Mar. 12, 2020.
(60) Provisional application No. 62/832,739, filed on Apr. 11, 2019.

(51) Int. Cl. A61K 8/67; A61K 8/04; A61K 8/34; A61Q 19/08

(52) U.S. Cl. CPC A61K 8/676; A61K 8/04; A61K 2800/87; A61Q 19/08; A61K 8/345

(57) ABSTRACT

Stable aqueous solutions containing high levels of ascorbic acid and water are described. These compositions have increased ascorbic acid stability due to the creation of solution media containing one or more polyhydric alcohols. The compositions are typically homogenous solutions, and can be adapted to be applied topically to the skin to impart appearance benefits thereto.

### FIELD OF INVENTION

The present invention relates generally to stable ascorbic acid compositions that are homogenous solutions. These solutions may be formulated into topical compositions. More specifically, the present invention relates to homogenous aqueous ascorbic acid compositions with increased stabilization due to one or more polyhydric alcohols with more primary hydroxyl groups than non-primary hydroxyl groups.

### BACKGROUND OF INVENTION

The application of various active ingredients to the skin can provide many benefits to skin health. For example, various active ingredients may help prevent radical-induced damage and/or increase collagen production. These active ingredients are often reactive and undergo various reactive processes which degrade topical utility of the ingredient over time. For example, ascorbic acid provides antioxidant protection, prevents photo-aging, and stimulates collagen production, but routinely reacts with various components in an environment (e.g., oxygen, photons, water). These reactions convert the active ascorbic acid to non-active reaction products of ascorbic acid (e.g., L-ascorbic acid 2-hydrogen sulfate, dehydroascorbic acid, etc.). Typically, the formation of this non-active reaction product of ascorbic results in a color change of the resultant composition to a more brown or orange color.

The use of ascorbic acid in topical compositions is further complicated by its solubility. Ascorbic acid is appreciably soluble in water, but as described above, is also unstable in water. For example, U.S. Pat. No. 6,299,889, hereby incorporated by reference in its entirety and specifically in relation to stabilization of ascorbic acid, stabilizes ascorbic acid compositions by minimizing the amount of water in solution. The media in U.S. Pat. No. 6,299,889 result in unstable ascorbic acid when the weight ratio of ascorbic acid to water is less than 1. On the other hand, the solubility of ascorbic acid in non-aqueous media is limited such that a solvent (e.g., ethanol) is required to dissolve limited amounts of ascorbic acid. Moreover, these non-aqueous media often result in non-aesthetically pleasing topical compositions which may be greasy or heavy feeling. For example, JP 2013-095691 A, hereby incorporated by reference and specifically in relation to its anhydrous formulations of ascorbic acid, discloses anhydrous compositions with 80% or more polyhydric alcohols. However, these formulations are plagued by solubility issues with ascorbic acid and do not have the aesthetic feel of aqueous compositions. Typically, polyhydric alcohols in these disclosures are used generically and without distinction due to that all polyhydric alcohols are generally known to have solubilization properties with ascorbic acid.

There is an unmet need for aesthetically pleasing compositions of stable ascorbic acid, and particularly, aqueous compositions with high concentrations of ascorbic acid and high water content. Even small increases in the water content of ascorbic acid aqueous solutions were known result in dramatically increased instability and/or aesthetic improvement of the resulting formulation.

The foregoing discussion is presented solely to provide a better understanding of the nature of the problems confronting the art and should not be construed in any way as an admission as to prior art.

### SUMMARY

In accordance with the foregoing objectives and others, aqueous compositions with increased stability of ascorbic acid are described. Through the use of specific polyhydric alcohols in compositions, the ascorbic acid to water ratio may be decreased resulting in compositions with increased aesthetics, increased ascorbic acid concentration, increased water content, and decreased ascorbic acid instability. Without wishing to be bound by theory, it is believed that compositions with increased primary hydroxyl content from polyhydric alcohols are able to stabilize the ascorbic acid in an otherwise unstable water environment. The aqueous compositions may comprise:

(a) ascorbic acid; and

(b) one or more polyhydric alcohols;

wherein the total number of primary hydroxyl groups in all of said one or more polyhydric alcohols is greater than the total number of secondary and tertiary hydroxyl groups in all of said one or more polyhydric alcohols; and
wherein the weight ratio of ascorbic acid to water is less than 1 (e.g., less than 0.9, less than 0.8, less than 0.7, less than 0.6, etc.). In some embodiments, the weight ratio of ascorbic acid to water is from 1:1 to 1:10 (e.g., 1:1 to 1:1:5, 1:1.5 to 1:10, 1:1 to 1:3, 1:1.5 to 1:5, 1:1.5 to 1:3, etc.). Such compositions may allow the composition to comprise more than 10% water by weight of the composition (e.g., more than 12%, more than 15%, etc.). In some embodiments, the composition may comprise between 5% and 15% ascorbic acid. In certain implementations, the composition may comprise between 12% and 70% water by weight of the composition and between 5% and 15% ascorbic acid by weight of the composition.

For example, the aqueous composition may comprise:

(a) between 1% and 20% ascorbic acid by weight of the composition;

(b) between 20% and 50% 1,3-propanediol by weight of the composition; and

(c) between 5% and 20% glycerin by weight of the composition;

wherein the total weight percentage of components (a)-(c) is less than 100% (e.g., less than 80%, etc.).

The aqueous environment also has utility in compositions comprising aqueous phases (e.g., water-in-oil emulsions, oil-in-water emulsions, etc.). In certain embodiments, the composition may comprise an aqueous phase, wherein said aqueous phase comprises:

(a) ascorbic acid; and

(b) one or more polyhydric alcohols;

wherein the number of primary hydroxyl groups in said one or more polyhydric alcohols is greater than the number of secondary or tertiary hydroxyl groups in said one or more polyhydric alcohols; and
wherein the weight ratio of ascorbic acid to water is less than 1 (e.g., less than 0.9, less than 0.8, less than 0.7, less than 0.6, etc.). In some implementations, the aqueous phase comprises

(a) between 1% and 20% ascorbic acid by weight of the aqueous phase;

(b) between 20% and 50% 1,3-propanediol by weight of the aqueous phase; and

(c) between 5% and 20% glycerin by weight of the aqueous phase;

wherein the total weight percentage of components (a)-(c) is less than 100% (e.g., less than 80%, etc.).

Methods of diminishing the dermatological signs of aging are also provided. Typically, these methods may comprise the topical application of a composition comprising:

(a) ascorbic acid; and

(b) one or more polyhydric alcohols;

wherein the total number of primary hydroxyl groups in all of said one or more polyhydric alcohols is greater than the total number of secondary and tertiary hydroxyl groups in all of said one or more polyhydric alcohols; and

wherein the weight ratio of ascorbic acid to water is less than 1 (e.g., less than 0.9, less than 0.8, less than 0.7, less than 0.6, etc.). In some embodiments, the weight ratio of ascorbic acid to water is from 1:1 to 1:10 (e.g., 1:1 to 1:1:5, 1:1.5 to 1:10, 1:1 to 1:3, 1:1.5 to 1:5, 1:1.5 to 1:3, etc.). In some embodiments, the method may comprise the topical administration of a composition comprising an aqueous phase with:

(a) ascorbic acid; and

(b) one or more polyhydric alcohols;

wherein the total number of primary hydroxyl groups in all of said one or more polyhydric alcohols is greater than the total number of secondary and tertiary hydroxyl groups in all of said one or more polyhydric alcohols; and
wherein the weight ratio of ascorbic acid to water is less than 1 (e.g., less than 0.9, less than 0.8, less than 0.7, less than 0.6, etc.).

Methods of increasing the stability of ascorbic acid in an aqueous solution are also described herein. In certain embodiments, the methods of increasing the stability of ascorbic acid comprise incorporating one or more polyhydric alcohols into said solution;

wherein the total number of primary hydroxyl groups in all of said one or more polyhydric alcohols is greater than the total number of secondary and tertiary hydroxyl groups in said one or more polyhydric alcohols. The one or more polyhydric alcohols may, for example, be mixed with a solution of ascorbic acid and water and agitated until a homogenous solution is obtained. In some embodiments, the polyhydric alcohols, water, and ascorbic acid may be mixed in any order with one other and agitated until a homogenous solution is obtained.

### DETAILED DESCRIPTION

Detailed embodiments of the present invention are disclosed herein; however, it is to be understood that the disclosed embodiments are merely illustrative of the invention that may be embodied in various forms. In addition, each of the examples given in connection with the various embodiments of the invention is intended to be illustrative, and not restrictive.

All terms used herein are intended to have their ordinary meaning in the art unless otherwise provided. All concentrations are in terms of percentage by weight of the specified component relative to the entire weight of the topical composition, unless otherwise defined.

As used herein, “a” or “an” shall mean one or more. As used herein when used in conjunction with the word “comprising,” the words “a” or “an” mean one or more than one. As used herein “another” means at least a second or more.

As used herein, all ranges of numeric values include the endpoints and all possible values disclosed between the disclosed values. The exact values of all half integral numeric values are also contemplated as specifically disclosed and as limits for all subsets of the disclosed range. For example, a range of from 0.1% to 3% specifically discloses a percentage of 0.1%, 1%, 1.5%, 2.0%, 2.5%, and 3%. Additionally, a range of 0.1 to 3% includes subsets of the original range including from 0.5% to 2.5%, from 1% to 3%, from 0.1% to 2.5%, etc. It will be understood that the sum of all weight % of individual components will not exceed 100%.

As used herein “substantially free” of an element indicates that the element is present in an amount that is inadequate to affect the degradation rate of ascorbic acid in the composition. For example, a composition may be substantially free of a component when it comprises less than 5% or less than 1% of that element.

As used herein, glycol is a 1,2-vicinal diol. Examples of vicinal diols include 1,2-propanediol, 1,2 butanediol, 1,2, hexanediol, and the like. It will be understood that dialkyl glycols such as ethylene glycol (2,2-oxydiethan-1-ol) or dipropylene glycol (a mixture of three isomeric compounds 4-oxa-2,6-heptandiol, 2-(2-hydroxy-propoxy)-propan-1-ol and 2-(2-hydroxy-1-methyl-ethoxy)-propan-1-ol) or polyethyleneglycols are not considered glycols herein as they are not 1,2-vicinal diols.

The present invention relates to solutions or compositions which may be adapted for topical application to skin. In these compositions, ascorbic acid is stabilized with in a specific medium to allow for high water content. As a suitable vehicle for the ascorbic acid, a stable composition is provided having desirably cosmetic qualities, including pleasant feel and appearance when applied to skin. These compositions are typically homogeneous solutions. The aqueous composition may comprise

(a) ascorbic acid; and

(b) one or more (e.g., two, three, four, five, etc.) polyhydric alcohols;

wherein the total number of primary hydroxyl groups in all of said one or more polyhydric alcohols is greater than the total number of secondary and tertiary hydroxyl groups in all of said one or more polyhydric alcohols; and
wherein the weight ratio of ascorbic acid to water is less than 1 (e.g., less than 0.9, less than 0.8, less than 0.7, less than 0.6, etc.). In some embodiments, more than 60% (e.g., more than 70%, etc.) of the hydroxyl groups in said one or more polyhydric alcohols are primary hydroxyl groups.

Suitable polyhydric alcohols include ethylene glycol, 1,2-propylene glycol, 1,3-propanediol, 1,2-butylene glycol, 2,3-butylene glycol, 1,4-butanediol, 2-methyl-2,4-pentanediol, diethylene glycol, dipropylene glycol, glycerin, trimethylolpropane, pentaerythritol, and sorbitol. In some embodiments, the compositions may comprise combinations of polyhydric such that the primary hydroxyl content of all the polyhydric alcohols is greater than the non-primary (e.g., secondary, tertiary, etc.) polyhydric hydroxyl content in the composition. In some embodiments, the composition may be free or substantially free of glycols (i.e., 1,2 vicinal diols). In some embodiments, the composition may be free or substantially free of C₄ or greater sugar alcohols (e.g., sucrose, glucose, fructose, lactose, maltose, cellobiose, arabinose, ribose, ribulose, galactose, rhamnose, raffinose, xylose, mannose, trehalose, mannitol, sorbitol, inositol, ribitol, galactitol, erythritol, xylitol, etc.). In certain implementations, the composition may comprise glycerin and/or 1,3-propanediol.

The one or more polyhydric alcohols are incorporated to achieve the increased stability of the ascorbic acid. For example, the one or more polyhydric alcohols may be present in an amount between 15% and 85% (e.g., between 20% and 50%, between 25% and 40%, etc.) of by weight of the composition (or aqueous phase). In corporation of the one or more polyhydric alcohols described herein will allow for higher water content of compositions without concomitant increases in ascorbic acid instability. For example, with the specific one or more polyhydric alcohols, the water may be present in the composition in an amount greater than 10% or greater than 10.5% or greater than 11% or greater than 11.5% or greater than 12% or greater than 12.5% or greater than 13% or greater than 13.5% or greater than 14% or greater than 14.5% or greater than 15% or greater than 15.5% or greater than 16% or greater than 16.5% or greater than 17% or greater than 17.5% or greater than 18% or greater than 18.5% or greater than 19% or greater than 19.5% or greater than 20% or greater than 20.5% by weight of the composition (or aqueous phase). In some embodiments, the weight ratio of the one or more polyhydric alcohols to water is between 10:1 to 1:1 (e.g., 5:1 to 1:1, etc.).

For example, the aqueous composition (or aqueous phase) may comprise

(a) between 1% and 20% ascorbic acid by weight of the composition (or aqueous phase);

(b) between 20% and 50% 1,3-propanediol by weight of the composition (or aqueous phase); and

(c) between 5% and 20% glycerin by weight of the composition (or aqueous phase);

wherein the total weight percentage of components (a)-(c) is less than 100% (e.g., less than 80%, etc.). In some embodiments, the composition comprises between 10% and 70% water by weight of the composition (or aqueous phase).

In some embodiments, the composition may comprise an organic carbonate. The organic carbonate may promote the solubility of the ascorbic acid when used in combination with the polyhydric alcohols and water. The organic carbonate may include linear and cyclic carbonates including dihydrocarbyl carbonates such as diethyl carbonate, diisopropyl carbonate, dibutyl carbonate, and the like. In some embodiments, the organic carbonate may be a five-member, six-membered, or seven-membered cyclic carbonate. In certain implementations, the organic carbonate is selected from ethylene carbonate, propylene carbonate (1,2-propylene carbonate), 1,2-butylene carbonate, 2,3-butylene carbonate, and mixtures thereof. The organic carbonate may be incorporated in amounts to help solubilize the ascorbic acid and result in stable solutions. In certain embodiments, the organic carbonate may be present in an amount between 0.1% and 25% by weight of the composition (or aqueous phase). In some embodiments, the organic carbonate is present in an amount between 1% and 10% or 1% and 12% by weight of the composition (or aqueous phase).

The compositions may further comprise monohydric alcohols. In some embodiments, the monohydric alcohol may be selected from methanol, ethanol, 1-propanol, 2-propanol, 2-methyl-1-propanol, 1-butanol, 2-butanol, 2-methyl-2-propanol, 2-propene-1-ol, 2-propyn-1-ol, 2-methoxy-1-ethanol, 1-methoxy-2-propanol, 2-methoxy-1-propanol, and mixtures thereof. The compositions (or aqueous phase) may comprise less than 50% ethanol or less than 40% ethanol or less than 30% ethanol by weight of the composition (or aqueous phase). In some embodiments the composition may comprise between 25% and 35% or between 15% and 35% or between 18% and 30% monohydric alcohol (e.g., ethanol) by weight of the composition (or aqueous phase).

The compositions may also comprise a thickening agent. For example, the composition may comprise one or more hydroxyalkyl cellulose thickening agents such as the lower hydroxyalkylcellulose derivatives such as hydroxyethyl cellulose and hydroxypropyl cellulose. The thickening agent may be present in an amount to provide aesthetically pleasing properties to the compositions. For example, the thickening agent may be present between 0.01% and 10% (e.g., between 0.01% and 5%, between 0.1% and 3%, between 0.01% and 1%, etc.) by weight of the composition (or aqueous phase).

The compositions may be formulated in a variety of product forms, such as, for example, a lotion, cream, serum, spray, aerosol, cake, ointment, essence, gel, paste, patch, pencil, towelette, mask, stick, foam, elixir, concentrate, and the like, particularly for topical administration. The composition is typically formulated as a lotion, cream, ointment, serum, or gel.

Thus, homogeneous and stable ascorbic acid containing solutions (or aqueous phases) of the present invention may have between 0.1% to about 16% ascorbic acid, 20 to 85% of one or more polyhydric alcohols having mostly primary hydroxyl groups, 0.3 to 25% organic carbonate, 10 to 30% water, and, optionally, 5 to 40% monohydric alcohol, and, optionally, 0.01 to 3% hydroxyalkyl cellulose by weight of the composition (or aqueous phase).

The compositions may be formulated in a variety of product forms, such as, for example, a lotion, cream, serum, spray, aerosol, cake, ointment, essence, gel, paste, patch, pencil, towelette, mask, stick, foam, elixir, concentrate, and the like, particularly for topical administration. The composition is typically formulated as a lotion, cream, ointment, serum, or gel.

Compositions comprising the aqueous phases described herein may be in the form of an emulsion. Non-limiting examples of suitable emulsions include water-in-oil emulsions, oil-in-water emulsions, silicone-in-water emulsions, water-in-silicone emulsions, wax-in-water emulsions, water-oil-water triple emulsions or the like having the appearance of a cream, gel or microemulsions. As used herein, the term “oil” includes silicone oils unless otherwise indicated. The emulsion may include an emulsifier, such as a nonionic, anionic or amphoteric surfactant, or a gellant, typically in an amount from about 0.001% to about 5% by weight.

The composition of the present invention may be prepared by blending the components such as ascorbic acid, the one or more polyhydric alcohols, one or more organic carbonates and water, and thereafter, optionally filtering the so prepared composition to remove insoluble particles, if present. However, in many embodiments, the higher water content afforded by the stabilizing media do not require filtration of insoluble ascorbic acid particles.

The ingredients may be mixed together at room temperature in a suitable, standard mixing vessel. Optionally, a monohydric alcohol, and/or a hydroxyalkyl cellulose and various other cosmetic ingredients may be added during blending. Such ingredients include emollients, moisturizers, colorants, fragrance, preservatives, and antioxidants. Lastly, ascorbic acid (e.g., ascorbic acid in powder form) may be added to the mixing vessel. The final composition may then be packaged in ordinary containers for distribution to consumers. Ascorbic acid, either in fine granular form or in ultrafine powder form, is commercially available from Roche Vitamins Inc., Hoffman-La Roche, Nutley, N.J.

In practice, the compositions of the present invention may be applied topically, preferably after cleansing the skin area to be affected with mild soap and warm water. After application, a standard moisturizing lotion or cream is optionally applied to the same skin area without affecting the efficacy of the ascorbic acid composition.

The present invention discloses stable compositions having ascorbic acid, which compositions are cosmetically elegant. When these compositions are topically applied to human skin, they produce skin appearance benefits including, but not limited to, improvements in luster, tone, elasticity, clarity, and a reduction in sagging, sallowness, photo damage and fine lines, wrinkles, and size of pores. The compositions of the present invention provide a stable environment for formulating ascorbic acid for a long shelf life. By doing so, these compositions also may avoid many packaging requirements for ascorbic acid stabilization in compositions with high levels of ascorbic acid.

### EXAMPLES

The following examples illustrate specific aspects of the instant description. The examples should not be construed as limiting, as the example merely provides specific understanding and practice of the embodiments and its various aspects.

### Example 1: Stability Measurements on Ascorbic Acid Aqueous Solutions

The stability of nine ascorbic acid aqueous solutions was measured. Solutions were prepared with the components as listed in Table 1. A control solution was also produced where the polyhydric alcohol was replaced with ethanol. Each of the solutions were prepared by mixing the ingredients at room temperature to produce homogenous solutions.

TABLE 1

| | Weight Percentage |
|---|---|
| Component | (w/w) (%) |
| Ascorbic Acid | 10.0 |
| Water | 19.5 |
| Polyhydric alcohol | 30.0 |
| Ethanol | 40.5 |

The polyhydric alcohol was varied in each solution with the indicated polyhydric alcohol in Table 2. The number of primary hydroxyl and non-primary hydroxyl groups as a molar percentage of the composition are shown in Table 2 as well. Since the dipropylene glycol is a mixture of three diols having the formula C₆H₁₄O₃, it was assumed each dipropylene glycol molecule has one primary hydroxyl group and one non-primary hydroxyl group (i.e., a 1:1:1 molar mixture of 4-oxa-2,6-heptandiol, 2-(2-hydroxy-propoxy)-propan-1-ol and 2-(2-hydroxy-1-methyl-ethoxy)-propan-1-ol. The solutions were placed in a 130° F. oven and the degradation of the ascorbic acid was measured daily. Each sample was heated in an environment without light to prevent any potential photoreactions of the ascorbic acid. The compositions were determined to have had appreciable degradation of the ascorbic acid once a visual change in color was identified.

TABLE 2

| | | Number of | |
|---|---|---|---|
| | | non-primary | |
| | Number of | hydroxyl groups | |
| | primary hydroxyl | per molecule | Day Degradation |
| Polyhydric | (mol —OH/100 g | (mol —OH/100 g | was Observed |
| alcohol | composition) | composition) | (day) |
| 1,3-propanediol | 0.79 | 0 | No Degradation |
| 1,3-butanediol | 0.33 | 0.33 | 17 Days |
| dipropylene glycol | 0.22 | 0.22 | 14 Days |
| polyethylene | 0.15 | 0 | No Degradation |
| glycol 400 | | | |
| glycerin | 0.65 | 0.33 | 19 Days |
| 1,2-propanediol | 0.39 | 0.39 | 10 Days |
| 2-methyl-2,4- | 0.25 | 0.25 | 7 Days |
| pentanediol | | | |
| sorbitol | 0.33 | 0.66 | 6 Days |
| control | | | 6 Days |

As can be seen, the polyhydric alcohol chosen has an effect on the stability of ascorbic acid in solution. The sorbitol solution, which has more non-primary hydroxyl groups than primary hydroxyl groups, was shown to confer no more stability than the control solution. Those solutions with the higher primary hydroxy group content had increased stability of ascorbic acid. Not all polyhydric alcohols behave identically with ascorbic acid in solution. Instead, only certain polyhydric alcohols are able increase the stability of ascorbic acid in an aqueous solution. In Table 2, polyethyelene glycol 400 is shown to have a low primary hydroxyl group content due to its high molecular weight (comparatively to the other polyhydric alcohols studied). However, it is notable that polyethylene glycol has two primary hydroxyl groups and no secondary or tertiary hydroxyl groups. Moreover, polyethyelene glycols are more hydrophobic than the other polyhydric alcohols examined, which may also have an effect on the ascorbic acid stability.

### Example 2: Stability Measurements on 1,3-Propanediol Solutions

The stability of propanediol solutions were measured following their production. The solutions were subjected to accelerated aging (placed in a dark oven at 120° F.) and the degradation was monitored by visual inspection. At the same time point, the solutions were removed from the accelerated aging atmosphere and visually inspected for degradation. Table 3 shows the results of these experiments.

TABLE 3

| 1,3-pro- | | | DM | Ascorbic | Propylene | |
|---|---|---|---|---|---|---|
| panediol | ethanol | glycerin | Water | Acid | Carbonate | Color |
| (% w/w) | (% w/w) | (% w/w) | (% w/w) | (% w/w) | (% w/w) | Change |
| 30.00 | 23.00 | 12.00 | 17.95 | 10.00 | 7.05 | Standard |
| 28.00 | 23.00 | 12.00 | 17.95 | 10.00 | 9.05 | Same |
| 32.00 | 23.00 | 12.00 | 17.95 | 10.00 | 5.05 | Same |
| 35.00 | 23.00 | 12.00 | 17.95 | 10.00 | 2.05 | Darker |
| 25.00 | 23.00 | 12.00 | 17.95 | 10.00 | 12.05 | Same |
| 30.00 | 25.00 | 12.00 | 17.95 | 10.00 | 5.05 | Same |
| 30.00 | 21.00 | 12.00 | 17.95 | 10.00 | 9.05 | Same |
| 30.00 | 28.00 | 12.00 | 17.95 | 10.00 | 2.05 | Same |
| 30.00 | 18.00 | 12.00 | 17.95 | 10.00 | 12.05 | Lighter |
| 30.00 | 23.00 | 14.00 | 17.95 | 10.00 | 5.05 | Same |
| 30.00 | 23.00 | 10.00 | 17.95 | 10.00 | 9.05 | Same |
| 30.00 | 23.00 | 17.00 | 17.95 | 10.00 | 2.05 | Same |
| 30.00 | 23.00 | 7.00 | 17.95 | 10.00 | 12.05 | Same |
| 30.00 | 23.00 | 12.00 | 15.95 | 10.00 | 9.05 | Slightly |
| | | | | | | Lighter |
| 30.00 | 23.00 | 12.00 | 19.95 | 10.00 | 5.05 | Same |
| 30.00 | 23.00 | 12.00 | 12.95 | 10.00 | 12.05 | Lighter |
| 30.00 | 23.00 | 12.00 | 22.95 | 10.00 | 2.05 | Slightly |
| | | | | | | Darker |

As can be seen, the enhanced ascorbic acid stability is not solely dependent on the solubility of ascorbic acid in polyhydric alcohols. For example, in these compositions, increasing 1,3-propanediol weight percentage to 35% (i.e., solubilizing more ascorbic acid) resulted in a faster degradation rate than when the 1,3-propanediol content was less than 32% by weight of the composition.

### Example 3: Stable Ascorbic Acid Solution

Stable ascorbic acid solutions with high water content may be produced by mixing the ingredients shown in Table 4 in variations of the indicated weight percentages.

TABLE 4

| Components | Weight Percentage |
|---|---|
| Ascorbic Acid | 10% |
| Water | q.s. |
| Glycerin | 10%-35% |
| 1,3-propanediol | 10%-35% |
| Ethanol | 15-30% |
| Propylene carbonate | 2-7% |
| Additional ingredients (e.g., butylated | ≥1% |
| hydroxytoluene, hydroxypropyl | |
| cellulose, etc.) | |

As various changes can be made in the above-described subject matter without departing from the scope and spirit of the present invention, it is intended that all subject matter contained in the above description, or defined in the appended claims, be interpreted as descriptive and illustrative of the present invention. Many modifications and variations of the present invention are possible in light of the above teachings. Accordingly, the present description is intended to embrace all such alternatives, modifications and variances which fall within the scope of the appended claims.

1. An aqueous composition comprising an aqueous phase of:
(a) ascorbic acid; and
(b) one or more polyhydric alcohols;
(c) more than 10% water by weight of the composition;
wherein the total number of primary hydroxyl groups in all of said one or more polyhydric alcohols is greater than the total number of secondary and tertiary hydroxyl groups in all of said one or more polyhydric alcohols; and
wherein the weight ratio of ascorbic acid to water is less than 1 and from 1:1 to 1:10; and
wherein the aqueous composition is packaged in a container.

2. The aqueous composition according to claim 1, wherein the weight ratio of ascorbic acid to water is from 1:1 to 1:10.

3. The aqueous composition according to claim 1, wherein said composition comprises more than 12% water by weight of the composition.

4. The aqueous composition according to claim 1, wherein said composition comprises between 1% and 30% ascorbic acid.

5. The aqueous composition according to claim 1, wherein more than 60% of the hydroxyl groups in said one or more polyhydric alcohols are primary hydroxyl groups.

6. (canceled)

7. The aqueous composition according to claim 1, wherein said one or more polyhydric alcohols comprise glycerin and/or 1,3-propanediol.

8. The aqueous composition according to claim 1, wherein said composition is free or substantially free of glycols and/or C₄ or greater sugar alcohols.

9. The aqueous composition according to claim 1, wherein said composition comprises between 15% and 85% of said one or more polyhydric alcohols by weight of the composition.

10. The aqueous composition according to claim 1 further comprising a monohydric alcohol and/or an organic carbonate and/or a thickening agent.

11. The aqueous composition according to claim 1, wherein the weight ratio of said one or more polyhydric alcohols to water is between 10:1 to 1:1.

12. An aqueous composition comprising:
(a) between 1% and 20% ascorbic acid by weight of the composition;
(b) between 20% and 50% 1,3-propanediol by weight of the composition; and
(c) between 5% and 30% glycerin by weight of the composition;
wherein the total weight percentage of components (a)-(c) is less than 100%.

13. The aqueous composition according to claim 12, wherein said composition comprises between 10% and 70% water by weight of the composition.

14. The aqueous composition according to claim 12, wherein said composition is a cream, lotion, ointment, or solution.

15. A composition comprising an aqueous phase, wherein said aqueous phase comprises:
(a) ascorbic acid; and
(b) one or more polyhydric alcohols; and
(c) more than 10% water by weight of the composition;
wherein the number of primary hydroxyl groups in said one or more polyhydric alcohols is greater than the number of secondary or tertiary hydroxyl groups in said one or more polyhydric alcohols; and
wherein the weight ratio of ascorbic acid to water is less than 1; and
wherein the composition is packaged in a container.

16. (canceled)

17. The composition according to claim 15, wherein said aqueous phase comprises between 10% and 70% water by weight of the aqueous phase.

18. A method for diminishing the appearance of dermatological signs of aging comprising topically applying to the skin in need thereof an aqueous composition according to claim 1.

19. The method according to claim 18, wherein said dermatological signs of aging are selected from sagging and fine lines and wrinkles.

20. A method of increasing the stability of ascorbic acid in an aqueous solution comprising more than 10% water by weight, the method comprising incorporating one or more polyhydric alcohols into said solution;
wherein the total number of primary hydroxyl groups in all of said one or more polyhydric alcohols is greater than the total number of secondary and tertiary hydroxyl groups in said one or more polyhydric alcohols.
